(19) United States
(12) Patent Application Publication
(10) Pub. No.: US 2024/0350752 A1
(43) Pub. Date: Oct. 24, 2024

(54) VALVED HOLDING CHAMBER WITH FLOW INDICATOR

(71) Applicant: Trudell Medical International Inc., London (CA)

(72) Inventors: Matthew Gravett, London (CA); Emmanuel Marbina, London (CA)

(73) Assignee: Trudell Medical International Inc., London (CA)

(21) Appl. No.: 18/640,419

(22) Filed: Apr. 19, 2024

Related U.S. Application Data
(60) Provisional application No. 63/461,093, filed on Apr. 21, 2023.

(51) Int. Cl. A61M 15/00; A61M 16/20

(52) U.S. Cl. CPC A61M 15/0086; A61M 16/208; A61M 2205/3334

(57) ABSTRACT

A valved holding chamber has an input end and an output end, which includes an inhalation valve and a user interface. The inhalation valve defines a first inhalation flow path between the input end and the user interface. A flow channel includes an inlet in flow communication with the holding chamber and an outlet in flow communication with the user interface downstream of the inhalation valve, wherein the flow channel bypasses the inhalation valve and defines a second inhalation flow path between the input end and the user interface. A flow indicator is positioned in the flow channel. A second flow channel, with a second flow indicator dispose therein, may define a third inhalation flow path between the input end and the user interface separate from the first and second inhalation flow paths. The inhalation valve, first flow indicator and second flow indicator may have different flow resistances.

This application claims the benefit of U.S. Application No. 63/461,093, filed Apr. 21, 2023 and entitled “Valved Holding Chamber With Flow Indicator,” the entire disclosure of which is hereby incorporated herein by reference.

### TECHNICAL FIELD

The present invention relates to a valved holding chamber having a flow indicator, and to systems and methods for the use and assembly thereof.

### BACKGROUND

The use of an aerosol medication delivery apparatus and system to administer medication in aerosol form to a patient's lungs by inhalation (hereinafter “aerosol delivery system(s)”) is well known in the art. As used herein, the term “substance” includes, but is not limited to, any substance that has a therapeutic benefit, including, without limitation, any medication; the terms “patient” and “user” include humans and animals; and the term “aerosol delivery system(s)” includes pressurized metered-dose inhalers (pMDIs), pMDI add-on devices, such as valved holding chambers, devices including a chamber housing and integrated actuator suited for a pMDI canister, nebulizers and dry powder inhalers. Examples of such aerosol delivery systems are disclosed in U.S. Pat. Nos. 4,627,432, 5,582,162, 5,740,793, 5,816,240, 6,026,807, 6,039,042, 6,116,239, 6,293,279, 6,345,617, and 6,435,177, the entire contents of each of which are incorporated herein by reference.

Conventional pMDIs typically have two components: 1) a canister component in which the medication particles and a propellant are stored under pressure in a suspension or solution form; and 2) a receptacle component, often referred to as an actuator boot, used to hold and actuate the canister and typically configured with a mouthpiece. The canister component typically includes a valved outlet from which the contents of the canister can be discharged. A substance is dispensed from the pMDI by applying a force on the canister component to push it into the receptacle component thereby opening the valved outlet and causing the medication particles to be conveyed from the valved outlet through the receptacle component and discharged from an outlet of the receptacle component. Air is drawn through the pMDI during inhalation, with the pMDI defining an airflow resistance. Upon discharge from the canister, the substance particles are “atomized” to form an aerosol.

PMDI holding chambers typically include a chamber housing with an input end and an output end. The mouthpiece portion of the pMDI receptacle is received in a backpiece located at the input end of the chamber housing. An example of such a backpiece is disclosed in U.S. Pat. No. 5,848,588, the entire contents of which are incorporated herein by reference. The output end of the chamber housing may include an inhalation valve or a containment baffle, or both, and a user interface, such as an adapter, a mouthpiece and/or a mask. The user interface may be coupled to the output end of the chamber housing or integrally molded with, and define in part, the output end of the chamber housing. Some holding chambers include an integrated receptacle for a pMDI canister thereby eliminating the need for a backpiece or other equivalent structure used to receive and hold the mouthpiece portion of a pMDI.

Pressurized metered dose inhalers (pMDIs) come in wide variety shapes and sizes, such that different pMDIs may have different airflow resistance characteristics. As the user inhales through a pMDI, the user creates a pressure drop between their mouth and atmospheric/ambient air in the user environment surrounding the pMDI. Depending on the resistance of the inhaler, air will flow through the pMDI at a given rate. The relationship between flow pathway resistance (R), pressure (P) and flow rate (Q) is defined by:

$$R = \frac{P}{Q} .$$

Given a constant pressure drop across any particular pMDI, the air flow rate through the pMDI is dictated by the pMDI's resistance; as the pMDI resistance increases, flow decreases, and vice versa.

Valved holding chambers may incorporate an audible flow indicator, often called a whistle, or harmonica, that may indicate an excessive inhalation flow rate during aerosol drug delivery. Excessive inhalation flow rates may lead to impaction of aerosolized drug particles in the upper airway of the user, thereby diminishing the amount of drug that may be delivered to the lungs. The whistles may be located in the backpiece of the holding chamber. If excessive inhalation flow rate occurs, then there is sufficient airflow passing through the whistle to actuate the whistle, for example by resonation, and make a sound, thereby alerting the user to alter their inhalation technique. Alternatively, some valved holding chambers may have a flow indicator, such as a whistle, built into the mouthpiece that is used for “positive” feedback, meaning that the whistle sounds when the user had achieved a minimum flow rate, as opposed to the “negative” feedback for excessive inhalation flow rate. In both embodiments, the whistle communicates directly with the ambient environment, meaning the flow through the flow indicator comes from the ambient environment surrounding the holding chamber. In this way, the flow indicator, e.g., whistle, and pMDI connect the chamber body to the ambient environment, or air, in a “parallel” arrangement, as shown schematically in FIG. 1. In this arrangement, the flow rate is dependent on the pressure and resistance of both the whistle and the pMDI:

$${Q}_{inhalation} = {Q}_{pMDI} + {Q}_{Whistle} = \frac{P}{{R}_{pMDI}} + \frac{P}{{P}_{Whistle}}$$

In order for the airflow rate through the whistle to be consistent at a given pressure, without regard to the type of pMDI being used, the expression would be:

$$\frac{P}{{R}_{pMDI 1}} + \frac{P}{{R}_{Whistle}} = \frac{P}{{R}_{pMDI 2}} + \frac{P}{{R}_{Whistle}}$$

Given that the pMDIs have differing resistances, however, the parallel arrangement does not allow for the whistle to trigger at a consistent flowrate because R_{pMD/1}≠R_{pMD/2}.

Many aerosol delivery systems may also lack a visual indication to alert a caregiver when a patient is inhaling, for example below the excessive inhalation flow rate threshold. In the case of a pMDI used in conjunction with a holding chamber, for example, it may be helpful for a caregiver to know if the patient is inhaling at a rate sufficient to open the inhalation valve to allow the aerosolized medication to exit the holding chamber, and/or to confirm that the device is being used properly. It may also be helpful to know when the patient is inhaling in order to coordinate the actuation of the pMDI with inhalation.

### SUMMARY

In one aspect, one embodiment of a valved holding chamber assembly includes a holding chamber adapted to contain a substance and having an input end adapted to receive a pressurized metered dose inhaler and an output end. An inhalation valve is disposed at the output end of the holding chamber and is moveable to an open position in response to an inhalation flow through the holding chamber and the inhalation valve. A user interface is coupled to the output end of the holding chamber in flow communication with the holding chamber when the inhalation valve is in the open position, wherein the inhalation valve defines a first inhalation flow path between the input end and the user interface. A flow channel includes an inlet in flow communication with the holding chamber between the input end and the output end and an outlet in flow communication with the user interface downstream of the inhalation valve, wherein the flow channel bypasses the inhalation valve, and wherein the flow channel defines a second inhalation flow path between the input end and the user interface. A flow indicator is positioned in the flow channel, wherein the flow indicator is actuatable in response to a flow rate in the flow channel during inhalation. In one embodiment, the flow indicator is configured as a whistle. In this embodiment, the flow channel is in flow communication with the holding chamber and the user interface, but is not in direct flow communication with the ambient environment.

In one embodiment, a second flow channel includes a second inlet in flow communication with the holding chamber between the input end and output end and a second outlet in flow communication with the user interface downstream of the inhalation valve. The second flow channel defines a third inhalation flow path between the input end and the user interface separate from the first and second inhalation flow paths. In this embodiment, the second flow channel is in flow communication with the holding chamber and the user interface, but is not in direct flow communication with the ambient environment. A second flow indicator may be positioned in the second flow channel, wherein the second flow indicator is actuatable in response to a second flow rate in the second flow channel during inhalation. In one embodiment, the inhalation valve has a first flow resistance, the first flow indicator has a second flow resistance, and the second flow indicator has a third flow resistance, wherein the second flow resistance is greater than the first flow resistance, and the first flow resistance is greater than the third flow resistance.

In another aspect, one embodiment of a valved holding chamber assembly includes a first flow path between an input end and a user interface, wherein the input end is configured for coupling to a medicament delivery device and wherein the first flow path includes an inhalation valve having a first flow resistance. A second flow path is defined between the input end and the user interface, wherein the second flow path bypasses the inhalation valve, wherein the second flow path includes a flow indicator having a second flow resistance, and wherein the second flow resistance is greater than the first flow resistance. In one embodiment, the flow indicator is configured as a whistle. In one embodiment, a third flow path is defined between the input end and the user interface, wherein the third flow path is separate from the first and second flow paths, wherein the third flow path has a second flow indicator having a third flow resistance, and wherein the first flow resistance is greater than the third flow resistance. Neither the second nor the third flow paths are in direct flow communication with the ambient environment.

The various aspects and embodiments provide significant advantages over other medication delivery systems and methods. For example, and without limitation, all of the external air from the ambient environment passes through the pMDI and into the chamber before flowing through any of the first, second and/or third flow paths to the user interface. In this way, the entire flow passes through the pMDI before splitting between the inhalation valve and the flow indicator, configured as a whistle in one embodiment, or before splitting between the inhalation valve and the second flow indicator. Since the inhalation valve and the flow indicator(s) each have a constant flow resistance, the flow indicator (e.g., whistle) may be configured to actuate at a specified flow rate, regardless of the pMDI being used with the valved holding chamber. In other words, the flow indicator(s) and inhalation valve are arranged in parallel, but the combination thereof is arranged in series with the pMDI, as shown in FIGS. 2A and B.

In addition, actuation (e.g., movement) of the second flow indicator allows users and caregivers to visually detect when the user is inhaling when using an aerosol delivery system such that the user or caregiver may be alerted to possible defects and/or improper inhalation, including without limitation an improper seal being formed between the patient's face and the aerosol delivery system's interface, such as a mask.

The foregoing paragraphs have been provided by way of general introduction, and are not intended to limit the scope of the following claims. The various preferred embodiments, together with further advantages, will be best understood by reference to the following detailed description taken in conjunction with the accompanying drawings.

### BRIEF DESCRIPTION OF THE DRAWINGS

FIG. 1 is schematic representation of a prior art valve holding chamber.

FIG. 2A is a schematic representation of a first embodiment of a valved holding chamber.

FIG. 2B is a schematic representation of a second embodiment of a valved holding chamber.

FIG. 2C is a schematic representation of a third embodiment of a valved holding chamber.

FIG. 3A is a side cross-sectional view of a first embodiment of a valved holding chamber.

FIG. 3B is an enlarged partial cross-sectional view of the valved holding chamber taken along line 3B in FIG. 3A.

FIG. 4 is a perspective view of a second embodiment of a valved holding chamber.

FIG. 5 is a top view of the valved holding chamber shown in FIG. 4.

FIG. 6 is a side view of the valved holding chamber shown in FIG. 4.

FIG. 7 is an end view of the valved holding chamber shown in FIG. 4.

FIG. 8 is a cross-sectional view of the valved holding chamber shown in FIG. 4 taken along line 8-8.

FIG. 9 is a graph showing the resistance pressure v. flow rate for various exemplary pMDI devices.

FIG. 10 is a cross-sectional view of one embodiment of a valved holding chamber with the various flow paths illustrated.

FIGS. 11A-D illustrate various whistle embodiments.

FIG. 12 is a partial, cross-sectional view of one embodiment of a valved holding chamber.

FIGS. 13A and B are partial cross-sectional view of one embodiment of a valved holding chamber with acoustical openings and valves.

FIG. 14 is an end view of one embodiment of the valved holding chamber showing the acoustical openings.

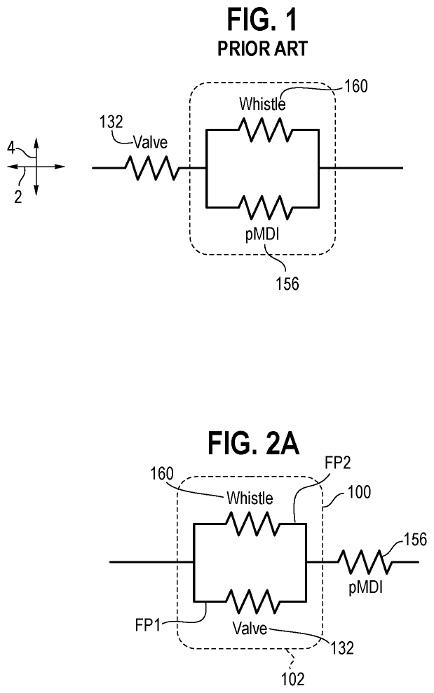

### DETAILED DESCRIPTION OF THE DRAWINGS AND PRESENTLY PREFERRED EMBODIMENTS

It should be understood that the term “plurality,” as used herein, means two or more. The terms “longitudinal” as used herein means of or relating to length or the longitudinal direction 2, for example between the opposite ends of the holding chamber or flow channel. The terms “lateral” and “transverse” as used herein, means situated on, directed toward or running from side to side, and refers to a lateral direction 4 orthogonal to the longitudinal direction. The term “direction” corresponds to an axis or line, rather than a vector. The term “coupled” means connected to or engaged with whether directly or indirectly, for example with an intervening member, and does not require the engagement to be fixed or permanent, although it may be fixed or permanent (or integral), and includes both mechanical and electrical connection. The terms “first,” “second,” and so on, as used herein are not meant to be assigned to a particular component so designated, but rather are simply referring to such components in the numerical order as addressed, meaning that a component designated as “first” may later be a “second” such component, depending on the order in which it is referred. For example, a “first” inlet may be later referred to as a “second” inlet depending on the order in which they are referred. It should also be understood that designation of “first” and “second” does not necessarily mean that the two features, components or values so designated are different, meaning for example a first inlet may be the same as a second inlet, with each simply being applicable to separate but identical components. As used herein: the term “substance” includes, but is not limited to, any substance that has a therapeutic benefit, including, without limitation, any medication; the terms “user” and “patient” includes humans and animals; and the term “aerosol delivery devices or system(s)” includes pressurized metered-dose inhalers (pMDIs), pMDI add-on devices, such as holding chambers, devices including a chamber housing and integrated actuator suited for a pMDI canister, nebulizers and dry powder inhalers. The phrase “flow communication” refers to two or more components or features constructed and arranged to provide for a flow of fluid (gas or liquid) therebetween, meaning for example a fluid may flow between an inlet and a holding chamber that are in “flow communication.” Two or more components or features may be in “flow communication” when a component, such as a valve, is open, but are not in flow communication when the component, such as the valve, is closed.

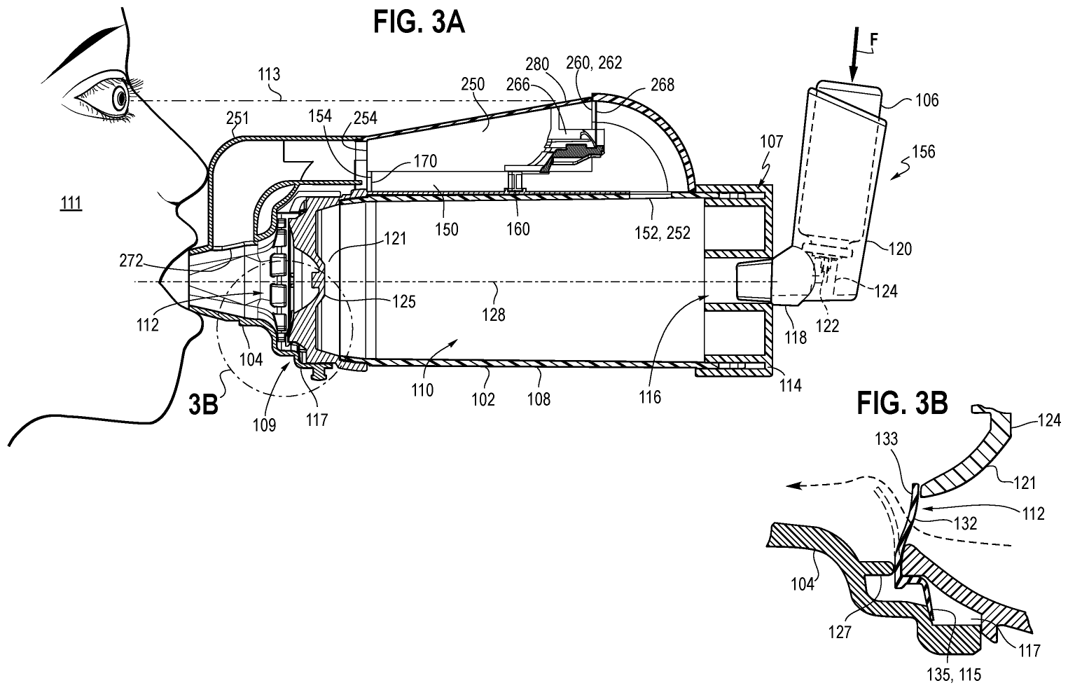

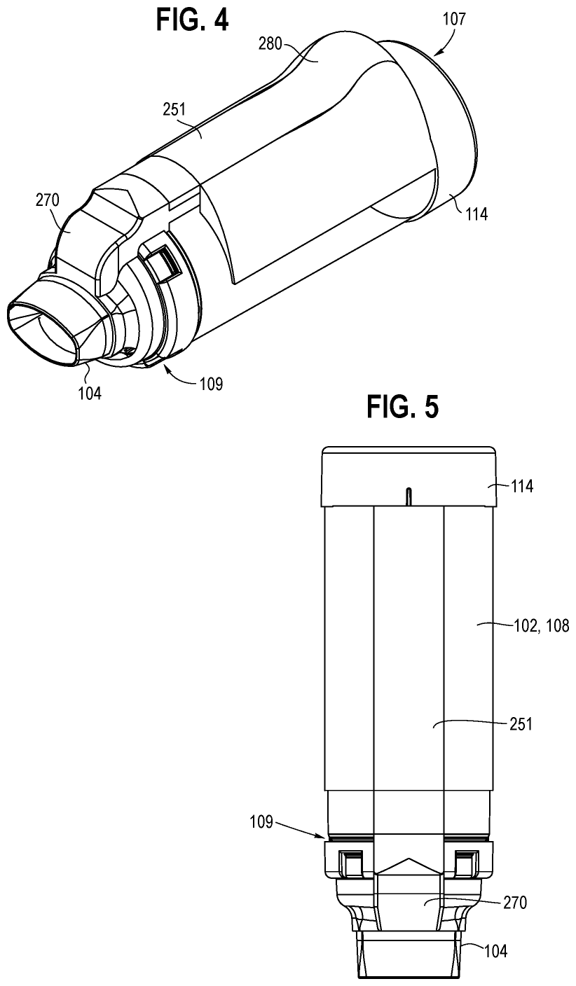

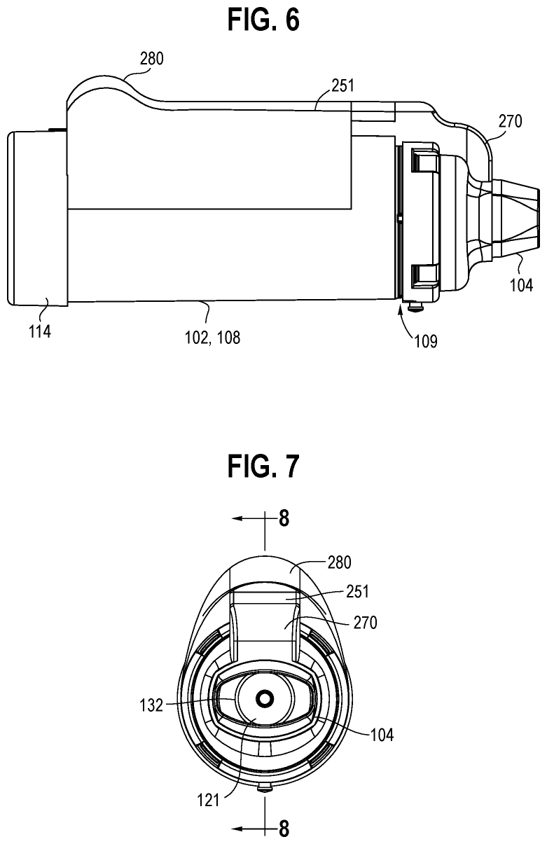

FIGS. 3A-8 show different embodiments of an aerosol delivery system 100. The system 100 includes a holding chamber 102 or conduit, a user interface 104, an inhalation valve 132 and a source of a substance, such as a pMDI canister 106, attached to a rear input end 107 of the holding chamber 102. The holding chamber 102 includes a chamber housing 108 that has a generally cylindrical shape with a generally circular cross-sectional shape that defines an interior volume 110 of space for receipt therein of aerosolized medication from the pMDI 156. A front output end 109 of the chamber housing includes an outlet 112, configured in one embodiment as an opening that is in fluid or flow communication with the interior volume 110 of space of the chamber housing 108. The user interface 104 may be configured as a mouthpiece, mask (nasal or oral/nasal), tube, or other suitable user interface, which is in fluid communication with the outlet opening 112. The inhalation valve 132 may be disposed over the opening to provide for one-way flow through the opening during inhalation but prevents back flow into the interior volume 110 during exhalation. The inhalation valve may be configured as any type of valve that permits one-way flow, including without limitation a flap valve, a duck-bill valve, an annular donut valve (i.e., valve having a central opening with a peripheral sealing edge), a center post valve, and/or a slit petal valve.

An exhalation valve 115 may be provided to allow for one-way flow to the ambient environment surrounding the system 100 during exhalation, but prevents air entrainment through an exhalation opening 117 during inhalation. The exhalation valve 115 may be configured as any type of valve that permits one-way flow, including without limitation a flap valve, a duck-bill valve, or an annular donut valve (i.e., valve having a central opening with a peripheral sealing edge), a center post valve, and/or a slit petal valve, and may be integrally formed with the inhalation valve as a single valve component.

The input end 107 of the chamber housing 108 is attached to, or includes, a detachable and flexible backpiece 114, or adapter, having an inlet 116, configured in one embodiment as an opening suited to receive the mouthpiece portion 118 of the pMDI receptacle 120, otherwise referred to as an actuator boot, that houses the pMDI canister 106. An exemplary pMDI 156 includes the canister 106 coupled to the receptacle 120. The canister 106 includes a valve stem 122 disposed in a well 124 in the bottom of the receptacle 120. The inlet 116 is in fluid communication with the interior volume 110. Examples of possible pMDI adapters and canisters to be used in conjunction with the holding chamber 102 are also described in U.S. Pat. Nos. 5,012,803, 5,012,804, 5,848,588 and 6,293,279, the entire contents of each of which is incorporated herein by reference. It should be understood that other aerosol delivery systems may have various one or more interior volumes fillable with an aerosolized medicament, with one or more outlets and one or more inlets communicating with the interior volume, including devices having a chamber housing and integrated actuator suited for a pMDI canister, nebulizers, dry powder inhalers and other such devices. The holding chamber 102 extends in the longitudinal direction 2, with the inlet 116 and outlet 112 being longitudinally spaced.

In one embodiment, a force F may be applied to the canister, thereby moving the valve stem 122 of the pMDI canister to discharge a predetermined dosage of medicament from the discharge end, e.g., mouthpiece portion 118, of the pMDI receptacle in aerosol form from the input end 107 into the interior volume 110 of the chamber housing 108. The aerosol medication particles within the interior volume 110 and chamber housing 108 are thereafter withdrawn through the outlet opening 112 at the output end 109 by having the user/patient 111 inhale through the interface 104.

The pMDI canister 106 contains a substance, preferably a medication suspension or solution under pressure. For example, the substance dispensed may be an HFA propelled medication suspension or solution formulation. Other medicaments, or medications, and propellants, such as CFC may also be used. It should be pointed out that while the described embodiments regard an aerosol delivery system for the delivery of an aerosolized medication from a pMDI, other aerosol delivery systems are contemplated that can be used within the spirit of the present invention. For example, it is contemplated that the flow indicator(s) may be incorporated with an aerosol delivery system such as existing ventilator systems, dry powder inhalers and nebulizers, in a manner similar to that described below. Examples of nebulizers that can be adapted to include such an indicator are disclosed in U.S. Pat. Nos. 5,823,179 and 6,044,841, the entire contents of which are incorporated herein by reference.

The present embodiments are not limited to the treatment of human patients. For example, it is contemplated that valved holding chamber may be configured with a use interface (e.g., mask) for administering medication to animals, including for example and without limitation equines, cats, dogs, etc.

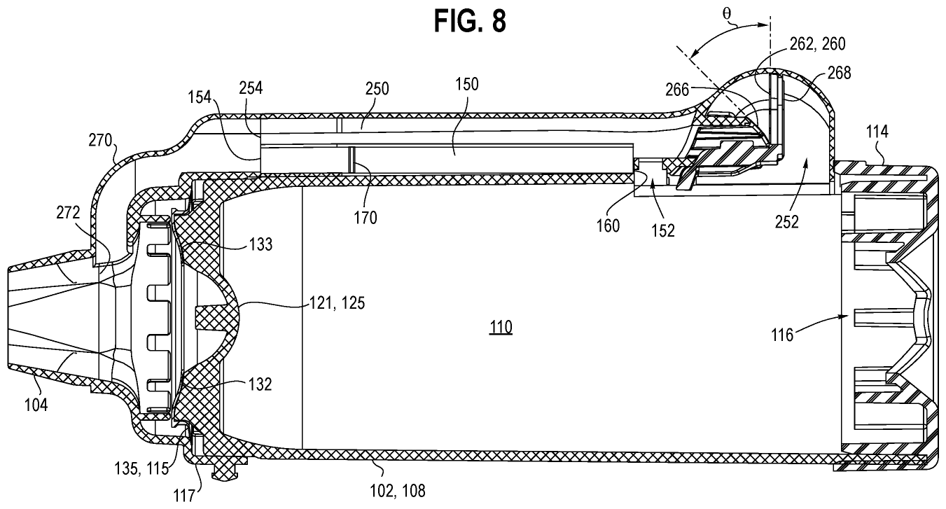

Referring to FIGS. 3A, 3B and 8, the output end 109 may include a containment baffle 121 positioned so as to partially block the opening 112. The baffle 121 reduces the velocity or flow rate or both of the aerosol medication particles flowing along the axis 128 of the chamber housing 108. A circular dome portion 125 of the baffle is aligned with the central axis 128 of the chamber housing 108 and is directly in line with the opening 112. Aerosol medication particles that have a flow path away from the central axis 128 tend to have a velocity that is lower than that of particles near to the axis 128. The baffle 121 may be configured with the dome portion 125, which reduces the forward, on-axis velocity and simultaneously acts as an impaction surface for on-axis projectile aerosol medication particles and so protects the valve 132. The dome portion 125 protrudes toward the input end 107. At the same time, the dome portion 125 allows slower moving aerosol medication particles to migrate towards the sides 130 of the chamber housing 108. It should be understood that the dome portion can alternatively be formed with a flat surface facing the input end, or a curved surface, for example a convex or concave surface.

As shown in FIGS. 3A, 3B, and 8, an annular valve 132 includes an inner inhalation valve portion 133 and an outer exhalation valve portion 135. The valve 132 is seated on the output end 109 of the holding chamber. The user interface 104, shown as a mouthpiece, includes retaining members 127 that clamp the valve 132 between the retaining members and the output end of the holding chamber. The valve may be made of a soft plastic, such as silicone or a thermoplastic elastomer.

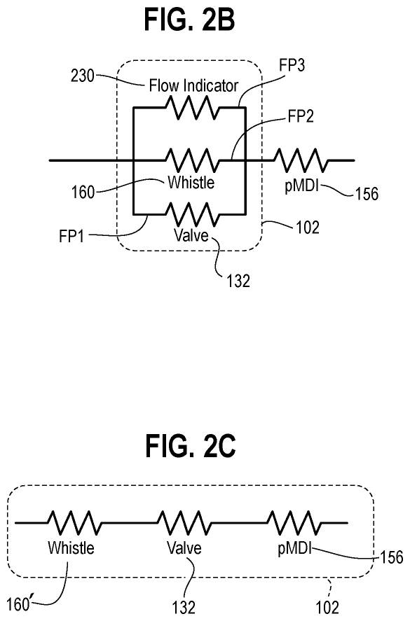

Referring to FIGS. 2A, 2B, 3 and 8, the aerosol delivery system 100, otherwise referred to as an aerosol delivery device, is configured with one or more flow channels 150, 250, which are preferably arranged on a top of the holding chamber such that the flow channels 150, 250 are visible to the user when using the device. It should be understood that the flow channels may be arranged, and extend radially from the holding chamber, at any location, including a bottom or side thereof. The flow channels may also be embedded radially inwardly from an outward surface of the holding chamber. In one embodiment, the flow channels are defined by an auxiliary housing 251 connected to the holding chamber 102. The auxiliary housing 251 may be integrally formed with the holding chamber, or formed separately and coupled thereto with tabs, fasteners, adhesives and the like.

The inhalation valve 132 is disposed at the output end 109 of the holding chamber and is moveable to an open position in response to an inhalation flow through the holding chamber 102. The user interface 104 is coupled to the output end 109 of the holding chamber in flow communication with the holding chamber when the inhalation valve is in the open position. In this way, the inhalation valve 132 defines a first inhalation flow path (FP1) between the input end, and the pMDI in particular, and the user interface, with reference to FIGS. 2A and 2B.

The auxiliary housing 251 defines a first flow channel 150 having an inlet 152 in flow communication with the holding chamber 102 between the input end 107 and the output end 109 and an outlet 154 in flow communication with the user interface 104 downstream of the inhalation valve 132, for example with the mouthpiece. The flow channel 150 is not in direct flow communication with the ambient environment, but rather is closed off or isolated from the ambient environment as the flow channel is in direct flow communication between the holding chamber 102 and the user interface 104. The flow channel 150 bypasses the inhalation valve 132 and defines a second inhalation flow path (FP2) between the input end, and in particular the pMDI 156, and the user interface 104. A flow indicator 160 is positioned in the flow channel 150. The flow indicator 160 is actuatable in response to a predetermined flow rate in the flow channel 150 during inhalation. In one embodiment, the flow indicator 160 is configured as an audible flow indicator, which may be a whistle. In one embodiment, the flow indicator 160′ may include a flexible strand disposed in the user interface 104, for example in the mouthpiece, in series with the inhalation valve 132 and pMDI 156, as shown in FIG. 2C. The strand may snap and wave like a flag during excessive inhalation.

Referring to FIGS. 2A, 2B, 3 and 8, the flow indicator 160 is actuatable when the flow rate in the flow channel 150 is greater than a threshold rate. The threshold flow rate in the flow channel 150 may include from 3 lpm-18 lpm (including for example 6 lpm), such that the flow from the flow channel 150 and the flow passing through inhalation valve 132 in combination are between 10 and 100 lpm, including for example 60 lpm, so as to provide indicia to the user that the inhalation rate is excessive.

In another embodiment, the inhalation valve incorporates, or is integrally formed with the flow indicator. For example, the inhalation valve may be configured as a duckbill valve having portions that flap excessively in response to excessive inhalation. Such embodiments may include a thin reed, formed from an elastic material, within the inhalation valve. The thin reed would flap excessively in response to excessive inhalation. This flapping would generate vibrations that can be felt by the user, and/or audible sound waves, as to alert the user of excessive inspiratory flow rate. In this embodiment, the valve 132 and flow indicator 160′ are in series, albeit integrally formed.

In one embodiment, an exhalation valve 170 may be disposed in the flow channel 150 defining the second inhalation flow path FP2, such that flow through the first flow channel 150 is one-way or unidirectional. The valve 170 may prevent moisture and residual drug contained in the mouthpiece area and exhaled breath from entering the flow channel where it could deposit and contaminate the flow channel. It also prevents exhaled air from flowing back through the flow channel into the chamber where it could cause remaining suspended aerosol drug that has not yet been inhaled to be pushed out of the chamber. In this aspect, it operates in the same manner that the inhalation valve 132 does when the user begins to exhale by ensuring air does not back-flow into the chamber and thereby affect the drug delivery. In one embodiment, the flow channel 150 may be configured with an exhalation flow indicator, for example a whistle, that is actuated in response to an excessive exhalation flow rate after drug inhalation, which may reduce the amount of drug delivered to the lungs. In this embodiment, the flow channel is not configured with an exhalation valve. In one embodiment, the flow indicator 160 may operate in response to both an inhalation and exhalation flow.

In one embodiment, two inhalation flow indicators, preferably one being a whistle 160 and the other being a visual indicator 260, may be positioned in series within a single flow channel 150. In this arrangement, as the user inhales, the visual indicator 260 moves forward in response to inhalation and reaches a maximum position as described with respect to other embodiments. However, the visual flow indicator 260 does not close off the channel 150 to flow, but rather allows flow to pass by and continue on through the channel and through the whistle 160. Alternatively, the whistle 160 may be positioned upstream of the visual indicator 260. In either position, when inhalation stops and exhalation begins, the visual indicator 260 returns to an at-rest position and closes off the flow channel 150 and in doing so, acts as an exhalation valve closing off the flow channel 150 to exhalation flow. In this embodiment the visual indicator 260 advantageously functions as both a visual indicator and an exhalation valve eliminating the need for the separate exhalation valve.

In one embodiment, the auxiliary housing 251 also defines a second flow channel 250 having a second inlet 252 in flow communication with the holding chamber 102 between the input end 107 and the output end 109 and a second outlet 254 in flow communication with the user interface 104 downstream of the inhalation valve 132. The first and second inlets may be defined by the same opening, as shown in FIG. 3, or different openings as shown in FIG. 8. Likewise, the first and second outlets may be defined by the same opening, or different openings. As shown in the embodiment of FIGS. 3A and 8, the outlets 154, 254 open into a passageway 270 having an outlet 272 in direct flow communication with the user interface 104. In one embodiment, the passageway 270 has a ninety-degree) (90° bend, or between a 30 and 60 degree bend, between the outlets 154, 254 and the outlet 272. In other embodiments, the passageway may have other orientations, or be straight, depending on the configuration and positioning of the user interface. The second flow channel 250 defines a third inhalation flow path (FP3) between the input end 107, and the pMDI 156 in particular, and the user interface 104 separate from the first and second inhalation flow paths FP1 and FP2. A second flow indicator 260 may be positioned in the second flow channel 250, wherein the second flow indicator is actuatable in response to a second flow rate in the second flow channel 250 during inhalation. In one embodiment, the second flow indicator 260 is configured as a visual flow indicator, defined by a moveable valve member 262. In one embodiment, the second flow indicator includes the valve member 262 moveable to a closed inhalation position, wherein the valve member engages or is seated on a valve seat 266 in response to the inhalation flow through the second channel. After the valve member engages the seat 266, the flow in the flow channel 250 is stopped. When the inhalation flow is stopped, the valve 262 returns to an at rest, or closed exhalation position, wherein the valve member engages or is seated on a valve seat 268. In the absence of any flow, or in response to an exhalation flow, the valve 262 is moved to the at rest position and prevents any exhalation flow from entering the holding chamber through the inlet 252. The valve member 262 is visible to the user and/or care giver through a viewing port 280, which may be defined by a transparent portion of the auxiliary housing 251. The viewing port 280 may be slightly elevated above the rest of the auxiliary housing, such that the user 111 engaged with the user interface 104 has a line of sight 113 to the viewing port 280, and the visual flow indicator 260 disposed therein. It should be understood that the flow channel 250 and flow indicator 260 are optional, meaning the holding chamber may be configured with only one flow channel 150 and flow indicator 160. In the embodiments shown in FIGS. 3A and 8, the viewing port 280, and flow indicator 260 are located at a distal end of the holding chamber 102, or closer to the input end 107 than the output end 109, with the inlets 152, 252 also located closer to the input end 107 than the output end 109. By positioning the flow indicator at a distal location, the visibility of the flow indicator is improved and limits or prevents the user from having to cross their eyes. In other embodiments, the flow indicator may be located closer to the output end 109 than the input end 107.

With the channel inlet 152 positioned at the distal end of the chamber, the amount of medication or drug that may enter the channel 150 is minimized. When the pMDI 156 is actuated, a medication plume advances towards the proximal end of the chamber, impacts the baffle 121, or domed portion 125, and then disperses or distributes throughout the chamber back towards the distal end. Once the user inhales through the chamber, which normally takes place shortly before or after the pMDI is actuated, the volume of air in the chamber, and therefore the suspended aerosolized drug, begins to evacuate through the inhalation valve 132 at the proximal end, with external air being pulled into the distal end of the chamber via the pMDI 156. Therefore, the channel 150, 250 intakes predominately new external air since the channel inlet 152, 252 is located at the distal end of the chamber, close to the pMDI 156. There may be some aerosolized medication that enters into the flow channel 150, 250, however the particle sizes are expected to be small considering most of the large particles would impact on the inside of the chamber closer to the proximal end/baffle 121. Also, due to timing of inhalation vs. pMDI actuation, the concentration of drug near the flow channel inlet 152, 252 is typically low. Therefore, the low quantity of small particle size medication that passes through the channel 150, 250 is more likely to make it through the flow channel and to the user's lungs in any event. Reducing the amount of drug entering the channel 150, 250 may help avoid drug build-up in the channel 150, 250, which may adversely impact the function of the whistle 160 and flow indicator 260.

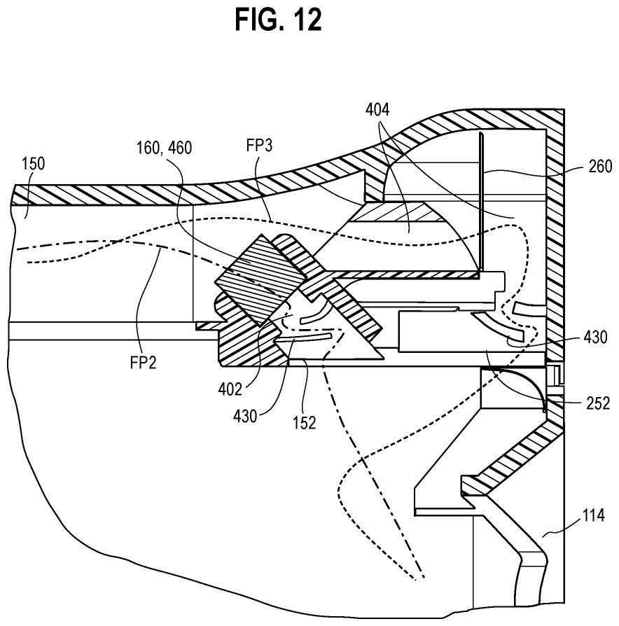

To further prevent any drug entering the flow channel 150, for example through channels 402, 404, or flow channel 250, a filter 420 may cover the inlet 152, 252. It should be understood that a single inlet 152 may feed both flow channels 402, 404. In one embodiment, a low flow resistance mesh filter 420 may be placed at the channel inlet 152, 252 to prevent drug from building up inside the channel 150, 250, 402, 404 and on the whistle 160, 460 and visual flow indicator 260. Such build-up my impact the performance of the whistle 160, 460 by affecting the airflow and resonance frequency of the reed(s) 462, or causing the visual flow indicator 260 to stick to any contacting surfaces. The filter 420 may be provided, or assembled, on an accessible area of the chamber for easier cleaning or replacement. The mesh filter 420 may be integrally molded with the holding chamber, or may be removable. The filter 420 may incorporate static properties to ensure the drug particles are attracted to and captured by the filter 420. In an alternative embodiment, shown in FIG. 12, a baffle 430 may be used instead of, or in combination with, the filter 420. The baffle 430 may be disposed at the inlet 152, 252 of each channel 150, 250, 402, 404, or a single baffle may be disposed at a single inlet feeding multiple channels 402, 404. The baffle 430 causes particles to impact on the baffle by creating a tortuous path through the flow path. The baffle 430 may be located within the chamber in area that is easy to clean.

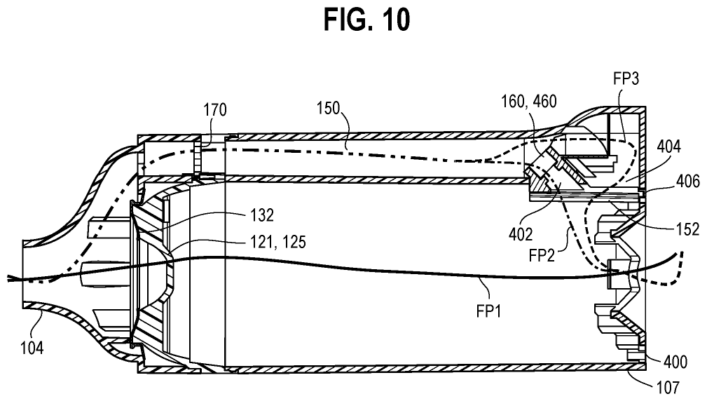

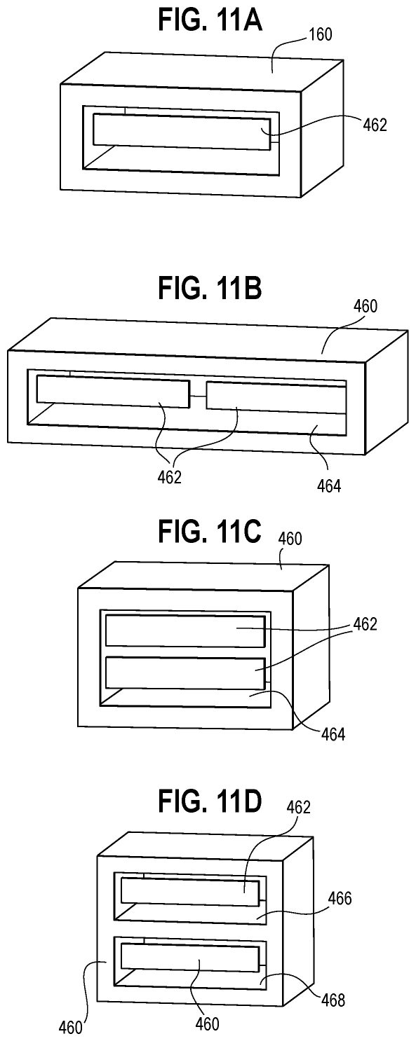

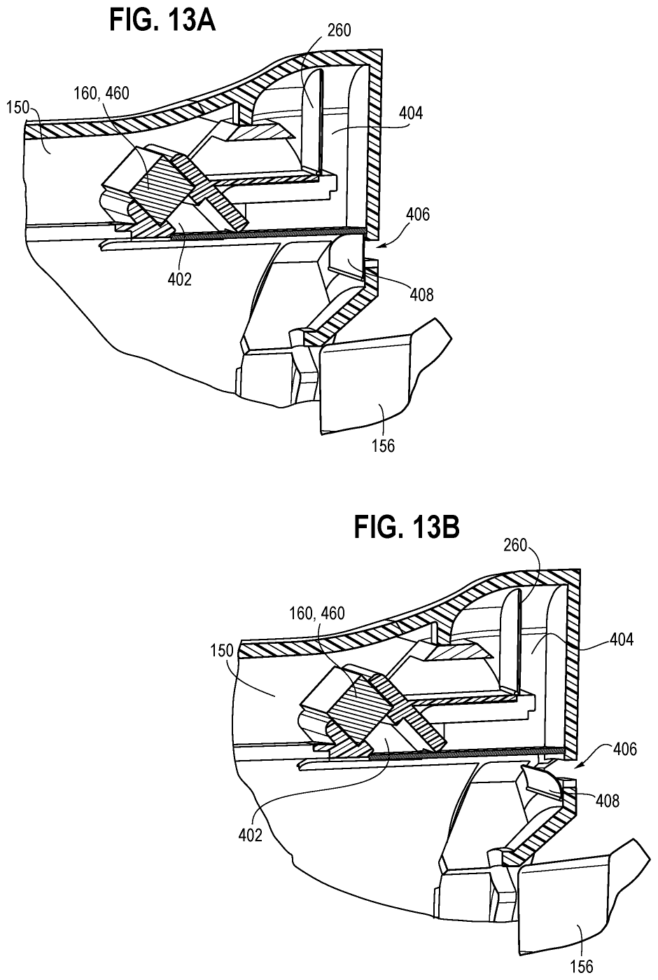

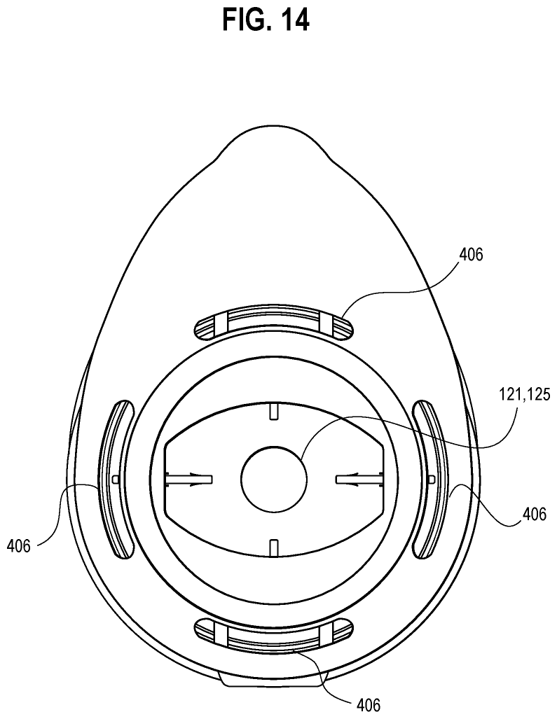

Referring to FIGS. 10, 11A-D, 13A and B and 14, a single flow channel 150 is provided, but with two flow paths FP2, FP3 having channels 402, 404 leading to the flow channel 150. An audible flow indicator 160, configured for example as a whistle, may be disposed in the channel 404 and flow path FP2. In order for the user or caregiver to hear the whistle, located in the flow channel 404, the sound may need to propagate through the channel 404, 150, into the chamber and through any gaps in the pMDI body and into the environment. In order to optimize the sound intensity of the whistle, one or more acoustical openings 406, otherwise referred to as sound vents or cutouts, may be formed in the adapter 114. The acoustical opening(s) 406 enhances sound propagation by eliminating the need for sound to propagate through the pMDI 156, i.e., flow channels formed therein, by providing the acoustical opening(s) 406 proximate the audible flow indicator 160, preferably located in the distal end of the chamber in one embodiment. The proximity reduces the distance the sound has to travel to reach the ambient environment and with fewer obstructions that may reduce the sound intensity. The audible flow indicator 160, e.g., whistle, may also be angled (i.e., having a vector intersecting the adapter) within the channel 404, as shown in FIG. 10, such that the maximum sound energy is directed towards the adapter and the sound vents to maximize the sound intensity to the ambient environment and the user. As it relates to flow, the acoustical opening(s) 406 may introduce a slight secondary flow path in addition to the air entering the chamber through the pMDI/adapter inlet. In use, this may have minimal impact including negligible effects on aerosolized drug delivery. Limited aerosolized medicament escapes from the chamber via the openings 406 since the maximum pressure of the chamber is equal to ambient pressure, and since the inhalation valve 132 prevents the user from exhaling into the chamber. Similarly, the pMDI 156 is open to atmosphere, or the ambient environment, and already introduces an open channel to atmosphere. Alternately, as shown in FIGS. 13A and B, one-way valves 408, shown as flexible flaps, may be disposed over the acoustical openings 406. The valves 408 are openable to the interior of the chamber in order to prevent air from escaping the chamber when the user is not inhaling. The valves 408, or flaps, will open on inhalation as shown in FIG. 13B, and stay open during excessive inhalation, allowing the sound of the whistle to escape from the chamber as the flaps 408 are open. When the user is not inhaling, in particular in the scenario where the pMDI 156 is actuated before the user starts inhaling, and when the pressure inside the chamber momentarily increases due to the expansion of the aerosol plume, which may cause an momentary outflow through an open sound vents, the flaps 408 would be closed and would prevent any outflow from inside the chamber, as shown for example in FIG. 13A. Alternately, instead of acoustical openings such as cutouts or vents, very thin wall sections may be added to the adapter which would maximize the release of sound energy and to minimize any dampening. Similarly, the walls of the flow channel 404, where the whistle 160 is located, may be designed in such a way as to optimize sound propagation through the walls and to the outside, ambient environment including the use of strategically located thin wall sections. Alternately, in both cases where thin wall sections are used to optimized sound transmission, different materials, specifically chosen for their acoustic properties, may be incorporated locally where it would have the most benefit on the release of sound energy to the outside environment and not let air to flow through.

The audible flow indicator 160, or whistle, may alert the user and/or caregiver that the inhalation rate it excessive, which has a negative impact on drug deposition. A single tone produced by a whistle may not be as alarming/off-putting as two (or more) simultaneous tones which are at least one semitone apart. As such, a plurality of audible flow indicators 160, or whistles, may emit sound frequencies that are one or more semitone apart and have the same trigger pressure. The use of a plurality of different whistles may result in a more alarming sound, which may have an advantageous effect on the user and/or caregiver, so as to thereby provide a more suitable warning that will be acted on. In an alternate embodiment, shown for example in FIGS. 11B-D, the whistle 460 may have two reeds 462 arranged in various configurations. In other embodiments, the use of first and second whistles with first and second tones being different by at least one semi-tone may be incorporated into other holding chambers and adapters not having a separate channel. Shown in the images are a standard single reed whistle 160 having a single reed 462, a dual reed whistle 460 where the reeds 462 are adjacent to one another and share the same resonance channel 464 in stacked and side-by-side configurations (FIGS. 11B and C), and a qual reed whistle where the reeds 462 have separate confined resonance channels 466, 468 (FIG. 11D).

In one embodiment, the inhalation valve 132 has a first flow resistance, the first flow indicator 160 has a second flow resistance, and the second flow indicator 260 has a third flow resistance, wherein the second flow resistance is greater than the first flow resistance, and the first flow resistance is greater than the third flow resistance. In this way, in one embodiment, the valved holding chamber system 100 includes a first flow path (FP1) between the input end 107 and the user interface 104, wherein the input end is configured for coupling to a medicament delivery device, or pMDI 156, and wherein the first flow path (FP1) includes an inhalation valve 132 having a first flow resistance. The second flow path (FP2) is defined between the input end 107 and the user interface 104, wherein the second flow path (FP2) bypasses the inhalation valve 132, wherein the second flow path includes a flow indicator 160 having a second flow resistance, and wherein the second flow resistance is greater than the first flow resistance.

In one embodiment, the third flow path (FP3) is defined between the input end 107 and the user interface 104, wherein the third flow path (FP3) is separate from the first and second flow paths, meaning the first, second and third flow paths are not in flow communication with each other, except at the respective inlets and outlets 152, 252, 154, 254, 272 communicating with the holding chamber 102 and the user interface 104, or in the combined channel 150 as shown in FIG. 10. The third flow path (FP3) has a second flow indicator 260 with a third flow resistance, wherein the first flow resistance is greater than the third flow resistance. In this way, the second flow indicator 260, when actuated, provides indicia that a threshold inhalation flow has been achieved, and communicates to the user 111 or care giver that a proper seal of the user interface 104, such as a mask, has been achieved together with an adequate inhalation flow. Conversely, the first flow indicator 160 is actuated when an excessive inhalation flow rate, greater than a predetermined threshold, has been achieved in the second flow path (FP2), such that the user 111 may adjust and lower the inhalation flow rate below the threshold. In this way, the first and second flow indicators 160, 260 provide the user with indicia about the upper and lower flow rate limits such that the user may adjust their inhalation accordingly.

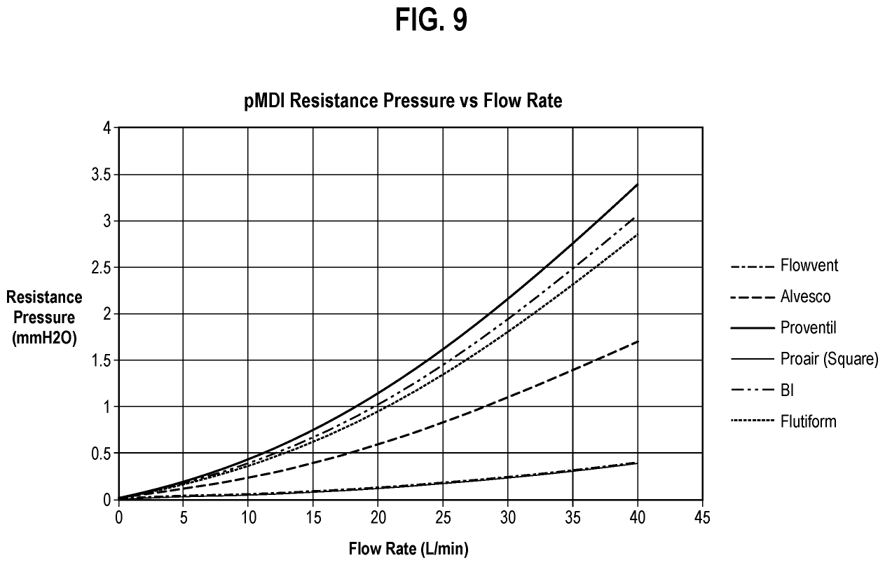

In operation, all air from the ambient environment passes through the pMDI 156 and into the holding chamber 102 before splitting, or bifurcating between the inhalation valve 132, or first flow path (FP1), and the flow indicator 150, or second flow path (FP2). The flow indicator 160 and the inhalation valve 132 each have a constant flow resistance, meaning the split of flow between the two flow paths, FP1 and FP2, will always be identical. Therefore, the flow indicator 160 may be actuated, or triggered, at a specified flow rate, regardless of the pMDI 156 in use. For example, as shown in FIG. 9, different pMDIs have different resistance pressures. In essence, the flow indicator 160 actuates independently of the pMDI 156 being used with the holding chamber 102. Upon inhalation, the air passes through the pMDI 156 and into the holding chamber 102 where it can take one of two paths-through the inhalation valve 132 (FP1), or through the flow channel 150 and through or past the flow indicator 160 (FP2). Once inside the holding chamber 102, the air will take the path of least resistance, with the majority of the airflow passing through the inhalation valve 132 and a portion passing through the flow channel(s) 150, 250. The path of least resistance is through the second flow channel 250, 404 defining the third flow path (FP3), if the system is configured with a second flow channel 250, 404. In one embodiment, the airflow reaches the flow indicator 260, or valve 262, at a trigger flow rate (between 0.5-7 lpm), thereby moving the flow indicator to indicate adequate inhalation flow. The user's inhalation flow rate is preferably between 15-30 lpm, but may be between 10 and 100 lpm. Once the trigger flow rate is achieved, the valve 262 seals against a valve seat 266, preventing further airflow through the flow channel 250, 404. With the flow channel 250, 404 closed, the airflow will pass from the pMDI 156 and through either the inhalation valve 132 or through the first flow channel 150. Since the second flow path (FP2) has a much higher resistance than the first flow path (FP1), most of the airflow passes through the inhalation valve 132 along the first flow path (FP1), while 5-30% (10% is preferred) of the user's inhalation flow passes through the second flow path (FP2) at higher flow rates, e.g., where the inhalation flow rate exceeds 60 lpm. In this context, lower flow rates describe any flow rate less than the flow rate required to actuate the inhalation valve 132. For reference, in one embodiment, the inhalation valve 132 acts in a binary fashion, such that once the valve 132 is actuated and opened, the resistance of the first flow path (FP1) suddenly decreases. The resistance of the first flow path (FP1) is constant, given that the user's inhalation flow rate remains above the threshold for higher flow rates.

In another embodiment, shown in FIG. 2C, the flow indicator 160 may be positioned in series with the inhalation valve 132 and pMDI 156. While the flow indicator 160 functions independently of the pMDI 156, the flow indicator may be positioned in the flow path to minimize any degradation of the flow indicator or delivery of the substance. For example, the flow indicator 160 may be disposed in or on the pMDI adaptor, not in flow communication with the ambient environment, but rather in communication with any flow in the holding chamber 102. In another embodiment, the flow indicator 160, configured for example as a whistle, may be disposed in the user interface 104, for example in series with the inhalation valve 132. Again, the flow through or past the flow indicator does not originate directly from the ambient environment, but rather comes through the inhalation valve 132 from the holding chamber 102 and through the pMDI 156.

With the above description of the structure of the aerosol delivery system 100, the operation of the system 100 can be readily understood. In particular, a patient 111 engages the user interface 104 and the patient or caretaker then presses the pMDI canister 106 within the pMDI receptacle 120 of the pMDI 156 attached to the backpiece 114 located at the input end 107 of the chamber housing 108, which causes the medication to be delivered in aerosol form to the opening 112 in the manner described previously.

At or just after the time of depressing the pMDI canister 106, the patient 111 inhales. During proper inhalation, the flow indicator 260 will pivot forward in response to the inhalation pressure and flow, for example by an angle θ of between 25° to 45°, and preferably 45°, and seal against the valve seat surface 266. The angle θ may be varied to accommodate the attributes of the patient, i.e., child v. infant. Note that the visual flow indicator 260 has minimal resistance, due to its size and shape, and will respond to low tidal volumes, which is ideal for infants (tidal volume of approximately 50 cc, flow rate approximately 5 lpm) and small children (tidal volume ranging from approximately 150 to 250 cc, flow rate approximately 12 lpm). The movement of the visual flow indicator 260 against the valve seat 266 creates a seal that prevents further entrainment of air from the holding chamber 102 through the flow channel 250, 404. A user and/or caregiver who directs his or her attention to the viewing port area 280 will be able to see the movement of the flow indicator 260 as the flow indicator 260 forms the seal and so will become aware that inhalation is occurring or has occurred. Also, during inhalation, the inhalation valve 132 will open thereby allowing the aerosolized medication to exit the chamber housing 108 through the opening 112 along the first flow path (FP1) upon which the medication flows along so as to eventually be inhaled by the patient. The flow indicator 260 is positioned in parallel with the inhalation valve 132, and outside of the first flow path (FP1), or medication dispensing pathway, and thus does not compromise medication delivery.

If the inhalation flow rate is excessive, or greater than a threshold flow rate, the flow indicator 160 in the first flow channel 150, 402 will be actuated, for example by sounding an alarm such as the whistle. In response, the user may decrease the inhalation flow rate below the threshold level such that the delivery of the medication to the lungs is optimized.

Once the patient exhales or ceases to inhale, the flow indicator 260 will pivot back to its original vertical position until it engages the valve seat 268. The resiliency of the flow indicator 260 pivots or biases the indicator to the at-rest position. Again, the patient 111 or caregiver who directs his or her attention to the viewing port area 280 will be able to see the return movement of the flow indicator 260 and so will become aware that inhalation has ceased. Besides alerting the caregiver that inhalation or exhalation is occurring or has occurred, the movement of the flow indicator 260 gives the caregiver confidence that, where the patient interface 104 includes a mask for example, a proper seal is formed between the patient's face and the mask, or if a mouthpiece, that a proper seal between the use's mouth and the mouthpiece has been achieved.

It should be understood that the flow channels 150, 250, 402, 404, together with the first and second flow indicators 160, 260, may be incorporated into other aerosol delivery systems such as dry powder inhalers and nebulizer systems, which include chamber housings, with the flow channel.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. A valved holding chamber assembly comprising:
a holding chamber adapted to contain a substance and comprising an input end adapted to receive a pressurized metered dose inhaler and an output end;
an inhalation valve disposed at the output end of the holding chamber and moveable to an open position in response to an inhalation flow through the holding chamber;
a user interface coupled to the output end of the holding chamber in flow communication with the holding chamber when the inhalation valve is in the open position, wherein the inhalation valve defines a first inhalation flow path between the input end and the user interface;
a flow channel comprising an inlet in flow communication with the holding chamber between the input end and the output end and an outlet in flow communication with the user interface downstream of the inhalation valve, wherein the flow channel bypasses the inhalation valve, and wherein the flow channel defines a second inhalation flow path between the input end and the user interface; and
a flow indicator positioned in the flow channel, wherein the flow indicator is actuatable in response to a flow rate in the flow channel during inhalation.

2. The valved holding chamber assembly of claim 1 wherein the flow indicator comprises a whistle.

3. The valved holding chamber assembly of claim 2 wherein the flow indicator is actuatable when the flow rate in the channel is greater than a threshold flow rate.

4. The valved holding chamber assembly of claim 2 further comprising an acoustical opening communicating between the chamber housing and the ambient environment proximate the whistle.

5. The valved holding chamber assembly of claim 4 further comprising a one-way valve communicating with the acoustical opening.

6. The valved holding chamber assembly of claim 2 wherein the whistle comprises first and second whistle emitting first and second tones, wherein the first tone varies from the second tone by at least one semi-tone.

7. The valved holding chamber assembly of claim 6 wherein the first and second whistles comprise first and second reeds respectively.

8. The valved holding chamber assembly of claim 1 wherein the inhalation valve comprises a first flow resistance and the flow indicator comprises a second flow resistance, wherein the second flow resistance is greater than the first flow resistance.

9. The valved holding chamber assembly of claim 1 wherein the flow channel comprises a first flow channel, the inlet comprises a first inlet, the outlet comprises a first outlet, the flow rate comprises a first flow rate, and the flow indicator comprises a first flow indicator, and further comprising a second flow channel comprising a second inlet in flow communication with the holding chamber between the input end and output end and a second outlet in flow communication with the user interface downstream of the inhalation valve, wherein the second flow channel defines a third inhalation flow path between the input end and the user interface separate from the first and second inhalation flow paths, and further comprising a second flow indicator positioned in the second flow channel, wherein the second flow indicator is actuatable in response to a second flow rate in the second flow channel during inhalation.

10. The valved holding chamber assembly of claim 9 wherein the inhalation valve comprises a first flow resistance, the first flow indicator comprises a second flow resistance, and the second flow indicator comprises a third flow resistance, wherein the second flow resistance is greater than the first flow resistance, and the first flow resistance is greater than the third flow resistance.

11. The valved holding chamber assembly of claim 10 wherein the second flow indicator comprises a valve member moveable to a closed position in response to the inhalation flow through the second channel.

12. The valved holding chamber assembly of claim 11 wherein the valve member is visible through a viewing port.

13. The valved holding chamber assembly of claim 9 wherein the user interface comprises a mask or mouthpiece.

14. The valved holding chamber assembly of claim 1 wherein the input end is configured to receive a mouthpiece portion of a pressurized metered dose inhaler.

15. The valved holding chamber assembly of claim 1 further comprising an exhalation valve disposed in the second inhalation flow path.

16. The valved holding chamber assembly of claim 1 further comprising a filter disposed in the inlet.

17. The valved holding chamber assembly of claim 1 further comprising a baffle disposed in the inlet.

18. A valved holding chamber assembly comprising:
a first flow path between an input end and a user interface, wherein the input end is configured for coupling to a medicament delivery device, wherein the first flow path comprises an inhalation valve having a first flow resistance; and
a second flow path between the input end and the user interface, wherein the second flow path bypasses the inhalation valve, wherein the second flow path comprises a flow indicator having a second flow resistance, wherein the second flow resistance is greater than the first flow resistance.

19. The valved holding chamber assembly of claim 18 wherein the flow indicator comprises a whistle.

20. The valved holding chamber assembly of claim 19 further comprising an acoustical opening communicating with the ambient environment proximate the whistle.

21. The valved holding chamber assembly of claim 21 further comprising a one-way valve communicating with the acoustical opening.

22. The valved holding chamber assembly of claim 20 wherein the whistle comprises first and second whistle emitting first and second tones, wherein the first tone varies from the second tone by at least one semi-tone.

23. The valved holding chamber assembly of claim 22 wherein the first and second whistles comprise first and second reeds respectively.

24. The valved holding chamber assembly of claim 18 wherein the flow indicator is actuatable when a flow rate in the second flow path exceeds a first threshold flow rate.

25. The valved holding chamber assembly of claim 18 wherein the flow indicator comprises a first flow indicator, and further comprising a third flow path between the input end and the user interface, wherein the third flow path is separate from the first and second flow paths, wherein the third flow path comprises a second flow indicator having a third flow resistance, wherein the first flow resistance is greater than the third flow resistance.

26. The valved holding chamber assembly of claim 25 wherein the second flow indicator comprises a valve member moveable to a closed position when a flow rate in the third flow path exceeds a second threshold flow rate.

27. The valved holding chamber assembly of claim 26 wherein the valve member is visible through a viewing port.

28. The valved holding chamber assembly of claim 24 wherein the user interface comprises a mask or mouthpiece.

29. The valved holding chamber assembly of claim 18 wherein the input end is configured to receive a mouthpiece portion of a pressurized metered dose inhaler.

30. The valved holding chamber assembly of claim 18 further comprising an exhalation valve disposed in the second flow path.

31. The valved holding chamber assembly of claim 18 further comprising a filter disposed in the inlet.

32. The valved holding chamber assembly of claim 1 further comprising a baffle disposed in the inlet.
